(19) United States
(12) Patent Application Publication
(10) Pub. No.: US 2009/0030494 A1
(43) Pub. Date: Jan. 29, 2009

(54) Method and devices for treatment of vulnerable (unstable) and/or stable atherosclerotic plaque by disrupting pathologic vasa vasorum of the atherosclerotic plaque

(76) Inventors: Christodoulos Stefanadis, Athens (GR); Nicholas Kipshidze, New York, NY (US)

Correspondence Address: MAYER BROWN LLP, P.O. BOX 2828, CHICAGO, IL, 60690, US

(21) Appl. No.: 11/412,040

(22) Filed: Apr. 25, 2006

Related U.S. Application Data
(60) Provisional application No. 60/674,997, filed on Apr. 26, 2005.

(51) Int. Cl. A61F 2/94

(52) U.S. Cl. 623/1.2; 623/1.42; 623/1.38; 128/898

(57) ABSTRACT

A drug-eluting stent is disclosed; together with various methods for treating atherosclerotic plaques and other cardiovascular diseases via intervention on vasa vasorum.

### BACKGROUND OF THE INVENTION

The present invention relates generally to local treatment of vulnerable and/or stable atherosclerotic plaque by disrupting pathologic vasa vasorum of the atherosclerotic plaque.

Atheroma and atherosclerosis date to the times of the ancient Egyptians (mummies had atherosclerosis and calcification of coronary arteries). Fallopius (1575) described a degeneration of the arteries into bone and at this time the process was felt to be a natural result of the aging process. Crell (1749) published a book regarding hardening of the coronary arteries. He felt that the inflammation noted within plaques produced pus that separated the muscular layer from the internal lining of the diseased artery. He noted that when the pus hardened it formed a scaly-like change on the lining of these vessels. At approximately this same time Boerhaave suggested that hardening of the arterial wall occurred when the small arteries that feed the muscular layer constricted and hardened (ossified), which is the first description of the vasa vasorum (the vessel within the vessel) directly involved in the angiogenic process.

Atherosclerosis is a systemic dysfunctional endothelial, focal occurring, chronic inflammatory, fibro-proliferative, prothrombotic, angiogenic, multifactorial disease of the arterial intima caused by the retention of modified low-density lipoproteins, hemodynamic, and reductive-oxidative (redox) stress.

There is no question that atherosclerosis is a systemic dysfunctional endothelial disease. It is focal in that lesions have a tendency to occur at predictable anatomic sites of the arterial tree. It predictably occurs at bifurcations, side branches, and opposite flow dividers at areas of low endothelial shear stress and turbulent blood flow. There is an orderly cephalad progression over time starting with the iliacs and progressing cephalad to the aorta, coronaries, carotids and cerebral vessels.

The PDAY (Pathobiological Determinants of Atherosclerosis in Youth) study and the Korean autopsy study revealed that the atheromatous process begins early in youth and young adulthood. By the fifth and sixth decades the devastating clinical effects of this malicious disease are witnessed and will increase as our population ages (as the “baby boom” generation transitions to the “senior boom” generation).

As the eccentric atheroma intima thickens, there is a relative ischemia of the vessel wall, which is a potent inducer of the adventitial angiogenic vasa vasorum (Vv). The chronic inflammation that runs concurrently serves to magnify this angiogenesis to the point that it appears to be uncontrolled as if a malignancy. The chronic inflammation (with its associated tissue factor) along with endothelial cell dysfunction contributes to the prothrombotic state of the atherosclerotic plaque. The retention of modified low-density lipoproteins is felt to be a key pathogenic event and possibly an absolute requirement for lesion development and progression. The hemodynamic stress is a prerequisite, as atherosclerosis does not develop within the venous system due to a low pressure-low shear stress environment. Also, pulmonary arteries do not develop atherosclerosis unless pulmonary hypertension is present.

There is an accelerated atherosclerosis (atheroscleropathy) associated with metabolic syndrome (MS), prediabetes (PD), and overt type 2 diabetes mellitus (T2DM). Plaque angiogenesis and intraplaque hemorrhage may be associated with unstable vulnerable plaques and contribute to plaque destabilization. See Moutlon, et al. , PNAS, Vol. 100(8):4736-4741 (Apr. 15, 2003); Moulton, et al., Circulation, 1999:99:1726-1732.

Angiogenesis in the setting of the vulnerable plaque is a double-edged sword. It is the body's natural protective response to ischemic injury of the vessel wall providing oxygen and metabolic nourishment as the intima undergoes a positive outward remodeling and thickening, while at the same time may contribute to plaque growth through the response to injury mechanism to intraplaque hemorrhage (IPH). As the numbers of these “malignant like” microvessels increase within the plaque, the numbers of IPH increase as a result and contribute to the instability of the atherosclerotic plaque.

Even though the IPH may be clinically silent, it may result in:

1. Rapid plaque growth due to increase in the size of the plaque, as well as the necrotic lipid core.

2. Serve as an angiogenic stimulus, thus auto-amplifying the continued vasa vasorum (angiogenic process) further increasing the chance for IPH.

3. Serve as an antigenic stimulus, thus auto-amplifying the continued intraplaque inflammatory response.

4. Activate the inflammatory macrophages at the shoulders of the plaque causing them to secrete their matrix metalloproteinases (MMPs) or collagenases causing a weakening and thinning of the protective fibrous cap as well as possible digestion of the fibrous cap resulting in erosion, fissuring, rupture, with platelet adhesion, aggregation, and ensuing thrombus formation with acute coronary syndrome.

There is, therefore, a need for a method to locally regress or stabilize plaque in the main blood supply. There is a further need for a treatment modality of vulnerable and stable atherosclerotic plaques, including the vasa vasorum of the atherosclerotic plaque.

### SUMMARY OF THE INVENTION

These needs and others may be met by the present invention having an aspect which is the administration of an anti-angiogenesis agent locally to an atherosclerotic plaque via a drug-eluting stent, local administration by local catheter delivery systems, intra-coronary intraluminal delivery via standard or specially delivery systems and systemic delivery or other suitable means in patients with atherosclerotic disease in different locations and different stages, stable and unstable forms. It is to be understood that both the foregoing general description and the following detailed description are not limiting but are intended to provide further explanation of the invention claimed.

### DETAILED DESCRIPTION

While the present invention is capable of embodiment in various forms, hereinafter is described an embodiment with the understanding that the present disclosure is to be considered as an exemplification of the invention, and is not intended to limit the invention to the specific embodiment illustrated.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of cardiology and pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value, yet cannot be given a precise limit. For example, in some cases, “about” or “approximately” may mean ±5%, or ±10%, or ±20%, or ±30% depending on the relevant technology. Also, the disclosure of ranges is intended as a continuous range including every value between the minimum and maximum values.

Vasa vasorum of an atherosclerotic vessel is the main blood supply to an atherosclerotic plaque. The present invention contemplates the use of anti-angiogenic agents to disrupt the blood flow and, hence, plaque growth or formation. This can be achieved by the use of anti-angiogenesis drugs locally administered to the affected area or by a drug that prevents or treats (by disruption, elimination, or reduction) pathologic vasa vasorum. These drugs include bevacizumab (Avastin®), Vitaxin®, angiostatin, endostatins and others. The pharmacological action of these agents is disruption of vasa vasorum of pathological tissue. This concept of cancer treatment and the drug angiostatin was introduced by Judas Folkman, M.D. and his team from Boston, Mass., USA.

In one embodiment of the present invention, such eluting stents are prepared. Once such stent, to be used as the drug delivery substrate, is the BiodivYsio stent delivery system (Biocompatibles, Ltd., U.K.), which is a laser cut, 316L stainless steel balloon-expandable stent coated with phosphorylcholine (PC), a naturally occurring biological substance. The biocompatible PC coating constitutes a 50-100 nm thick double double layer of synthetic PC coating that is able to absorb a drug via a sponge-like mechanism. The method of impregnating the PC-coated stent comprises the steps of:

1. Immersing the stent into a solution or suspension of bevacizumab (Avastin®), which was mixed according to the manufacturer's instructions (i.e., 25 mg/ml. The stent is immersed for at least about 5 minutes.

2. After removal of the stent from the solution and allowing it to dry for at least about 1 minute, about 10 microliters of the same drug solution is pipetted onto the stent. The PC polymer acts like a sponge in absorbing the drug solution/suspension.

3. The stent is again allowed to air dry for 1 minute. Then the above process is repeated.

After air-drying for about 5 minutes, the stent is immediately deployed into the patient's vessel as is known in the art. About 0.01 to about 10.0 micrograms/mm² of the drug can be impregnated using this method. Any anti-angiogenic agent or agent that prevents or treats (by disruption, elimination, or reduction) pathologic vasa vasorum (e.g., Vitaxin®, bevacizumab, angiostatin, endostatin), or a combination thereof, can be employed in the above process. The amount of drug impregnated to the stent can be at least one of the following approximate amounts:

About 0.01; 0.05; 0.1; 0.5; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9; 3.0; 3.1; 3.2; 3.3; 3.4; 3.5; 3.6; 3.7; 3.8; 3.9; 4.0; 4.1; 4.2; 4.3; 4.4; 4.5; 4.6; 4.7; 4.8; 4.9; 5.0; 5.1; 5.2; 5.3; 5.4; 5.5; 5.6; 5.7; 5.8; 5.9; 6.0; 6.1; 6.2; 6.3; 6.4; 6.5; 6.6; 6.7; 6.8; 6.9; 7.0; 7.1; 7.2; 7.3; 7.4; 7.5; 7.6; 7.7; 7.8; 7.9; 8.0; 8.1; 8.2; 8.3; 8.4; 8.5; 8.6; 8.7; 8.8; 8.9; 9.0; 9.1; 9.2; 9.3; 9.4; 9.5; 9.6; 9.7; 9.8; 9.9; 10.0 micrograms/mm².

In another embodiment, a mobile system that can be operated in a lab that allows for a non-polymer based stent coating process may be used to coat the drug on the stent.

In another embodiment, the foregoing stent is also coated with one or more anti-proliferative and/or anti-inflammatory agents such as sirolimus (rapamycin), everlomius, dexamethasone, biolimus, ABT 75, paclitaxel, or their salts, prodrugs, derivatives and analogues. These agents are impregnated onto, or otherwise adhered to, the stent together with one or more anti-angiogenic agents in an amount, as is known in the art, sufficient to prevent or inhibit atherosclerotic plaque formation. For example, the amount of antiproliferative and/or anti-inflammatory drug can be similar to that of currently available drug coated stents. In another embodiment the dose may be lowered in view of synergistic or additive effects of the above drug combination.

Any stent may be employed in the present invention suitable for drug impregnation, adsorption or absorption, and the present invention is not limited to the BiodivYsio stent. In another embodiment, for example, the stent may be a different balloon-expandable stent. In a further embodiment, the balloon-expandable stent may be made of stainless steel, cobalt chromium or other metals or alloys. In another embodiment, the stent is constructed of a polymer, or a biodegradable alloy, biodegradable polymer or other material. The balloon-expandable stent may be a solid sheet of material. In a further embodiment, the stent wall is perforated or a mesh. In another embodiment, the balloon-expandable stent has at least one smooth end. In a further embodiment both ends of the stent are smooth. In another embodiment the stent is a covered stent.

In some embodiments the balloon-expandable stent may have a length of about 5 mm to about 150 mm. The balloon-expandable stent may have a diameter of about 2 mm to about 12 mm. For example, the diameter may be about 2 mm, about 3 mm, about 4 mm, about 5 mm, about 6 mm, about 7 mm, about 8 mm, about 9 mm, about 10 mm, about 11 mm, or about 12 mm.

In other embodiments the stent may be a self-expandable stent. In another embodiment the self-expandable stent is composed of a metal, an alloy, nitinol, a polymer, a biodegradable alloy, biodegradable polymer or other material, or a combination thereof. The self-expandable stent may be a solid sheet of material. In a further embodiment, the stent wall is perforated or a mesh. In another embodiment, the self-expandable stent has at least one smooth end. In a further embodiment both ends of the stent are smooth. In another embodiment the stent is a covered stent.

In some embodiments the self-expandable stent may have a length of about 5 mm to about 150 mm. The self-expandable stent may have a diameter of about 2 mm to about 12 mm. For example, the diameter may be about 2 mm, about 3 mm, about 4 mm, about 5 mm, about 6 mm, about 7 mm, about 8 mm, about 9 mm, about 10 mm, about 11 mm, or about 12 mm.

In another embodiment, the anti-angiogenic drug or drug that prevents or treats (by disruption, elimination, or reduction) pathologic vasa vasorum is administered locally to the plaque area via an irrigator catheter. In another embodiment, the drug is delivered into periadventitial areas via nipple, needle or needles comprising catheters. In a further embodiment, the drug is delivered intramurally via special local delivery catheter system, including but not limited to pressure mediated diffusion systems, convection driven delivery systems, iontophoretic-mediated diffusion systems, and active injector systems. In still another embodiment, the drug is delivered intraluminally into the lumen of a diseased artery via an infusion local delivery catheter system or other suitable system. In other embodiments a balloon catheter is coated with the agent to deliver the agent to a target area. A dose of the drug, e.g., about 0.01; 0.05; 1.0; 2.0; 3.0; 4.0; 5.0; 6.0; 7.0; 8.0; 9.0; or 10.0 micrograms, is administered by bathing the affected area or by direct, local injection.

In another embodiment, the agent is administered systemically. In some such embodiments, systemic administration is nanoparticle-based. In another embodiment, the above drug coated stent is used with the above local or systemic administration.

In another embodiment, a catheter is employed to deliver the drug-eluting stent, which also has temperature sensors to measure the temperature of the plaque before and after the procedure. Alternatively, an injection catheter (without stenting) can be employed comprising temperature sensors.

In yet another embodiment, the atherosclerotic vessel is denervated by chemical, laser or mechanical means (e.g., botox, micro-knives, other toxic compounds, laser, ultrasound, etc.) to avoid recoil (shrinking) of the artery after balloon angioplasty and/or growth of the scar tissue after interventions.

In a further embodiment, a combination of at least two of denervation, stent placement and drug administration (systemic and/or local) may be used.

All of the foregoing devices and methods can be employed to treat a variety of cardiovascular diseases, e.g., acute coronary syndrome, atherosclerosis, and metabolic syndrome.

Those skilled in the art will appreciate that numerous other embodiments and modifications are contemplated by the present invention. The above description of embodiments is merely illustrative and not intended to limit the scope of the present invention. The patents, literature, and references cited herein are incorporated by reference in their entireties.

### EXAMPLES

The following Examples are provided for illustrative purposes only and are not to be interpreted as limiting the scope of the present invention in any manner.

### Example 1

Twenty patients with recent (less than a month) acute coronary syndrome (ACS) were selected. The patients all suffered from two-vessel coronary artery disease (culprit and non-culprit). The non-culprit lesion stenosis ranged from 60-70%. Mean age of the patients was 64 years of age, and the mean stenosis was 70%.

Percutaneous transluminal coronary angioplasty (PTCA) was used in the culprit vessel. A bevacizumab (Avastin®) eluting BiodivYsio (Biocompatibles Ltd., London, UK) stent was deployed in the non-culprit vessel. The mean stent diameter was 2.825 mm, and the mean stent length was 13.55 mm.

The study is ongoing; however, a follow-up examination was performed with the patients after three months. At that time, all patients had survived, and there had been no incidence of any major adverse effects including myocardial infarction nor had there been any need for target lesion revascularization or target vessel revascularization. Angiographic and intravascular ultrasound (IVUS) follow-up examinations were scheduled for six months and one year after implantation.

### Example 2

BiodivYsio stent delivery systems (Biocompatibles Ltd., London, UK) coated with phosphorylcholine (PC) were impregnated with bevacizumab in a three step process. First, the stent was immersed into a solution of 4 ml bevacizumab (Avastin®, 25 mg/ml, Roche) for 5 minutes. After removal of the stent from the solution and allowing it to dry for 1 minute, a second step by 10 microliters of the same solution was pipetted onto the stent and absorbed by the PC polymer. The stent was again allowed to dry for 1 minute, and the process was repeated, but with 5 minutes of air-drying.

Ten New Zealand rabbits with average weight 3.8±0.4 kg; range, 3.3 to 4.2 kg, were used in the study. All animals consumed an atherogenic diet (certified Purina Rabbit Chow, 5322, 95% with 0.3% cholesterol and 4.7% coconut oil, Research Diets) for three weeks to induce atheroma formation according to previous studies.

Both an uncoated BiodivYsio stent and a coated stent as discussed above were delivered in the middle segment of the 2 iliac arteries through the right carotid artery by a 5 Fr sheath. Eluting and non-eluting stents were 2 mm in diameter and the stent length was 7 mm (2 stents), 10 mm (12 stents) and 18 mm (6 stents). The balloon-expanded stent to artery ratio was intended to be 1.2:1 in all stents. Post-dilation was required in 12 stents. A final angiogram was performed to confirm the optimal expansion of the stents. All angiograms before and after the implementation were recorded in a video tape. At the end of the procedure, the angioplasty equipment was withdrawn, the carotid artery ligated, and the animal allowed to recover. The animals were continued to be in atherogenic diet. After stent implementation, the animals were treated with aspirin and clopidogrel. At 28 days a follow-up angiogram was performed after accessing the left common carotid artery and the animals were then euthanized with an intravenous overdose of thiopentone.

Stent implantation in all animals was successful, there were no problems related to the intervention, and all interventional devices were deployed successfully. All vessels were angiographically patent at the end of the procedure. There was no acute or subacute thrombosis. All animals survived to 28 days. Table 1 shows the diameter of the arterial segments at baseline and the balloon to artery ratios achieved in each group. Both the arterial diameter and the balloon to arty ratios were very similar for each group.

TABLE 1

| | Bevacizumab | Control | P value |
|---|---|---|---|
| Artery diameter (mm) | 2.01 ± 0.02 | 2.02 ± 0.03 | 0.81 |
| Stent length (mm) | 12.1 ± 4.1 | 12.1 ± 4.1 | 0.99 |
| Balloon/artery ratio | 1.20 ± 0.01 | 1.20 ± 0.01 | 0.99 |
| MLD after (mm) | 2.21 ± 0.01 | 2.22 ± 0.02 | 0.87 |
| MLD follow-up (mm) | 2.070 ± 0.02 | 2.05 ± 0.02 | 0.85 |

Iliac artery lumen diameters before and after stent placement were similar in all stent treatment groups. At euthanasia, stent diameters were similar in all groups. All stents were angiographically patent at the time of euthanasia without aneurysm formation (FIG. 1). Additionally gross pathologic analysis did not show any evidence of vascular necrosis.

Angiographic Assessment The diameter of the arterial segments in eluting and control stents (2.01±0.02 vs. 2.02±0.03, p=0.81) and the balloon to artery ratios achieved in each group (1.20±0.01 vs. 1.20±0.01, p=0.99) were similar (Table 1).

Iliac artery minimal lumen diameters immediately after stent placement were similar in both stent treatment groups (eluting: 2.21±0.01 vs. control 2.05±0.02, p=0.85). All stents were angiographically patent at the time of euthanasia without aneurysm formation.

### Histological Analysis

Mean injury scores on day 28 were similar for the arteries that received either the control or the coated stents (1.22±0.40 and 1.32±0.40, respectively; p=0.71). Although a modest stent neointima formed in all rabbit arteries, stent neointimal area was 38% less than in those rabbits that received the coated stent than in those with the control stent (0.60±0.12 versus 0.97±0.16 mm²; p<0.01).

Neovascularization was significantly decreased in the bevacizumab-eluting stents (1.4±1.7 neovessels per mm² plaque) versus the control group (13.9±2.9 neovessels per mm² plaque; p<0.01), as were local inflammation where bevacizumab-eluting stents demonstrated significantly lower macrophage recruitment per cross section (33.8±4.1 cells per cross section) relative to the control group (56.4±4.1 cells per cross section; p<0.01).

As such, the preliminary results of the study show that bevacizumab-eluting stent implantation in atheromatic rabbit iliac arteries is feasible and safe, and the immediate and late angiographic results demonstrated that there is no increased thrombogenicity compared to the control group.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles. All patent and literature references are hereby incorporated by reference as if fully set forth herein.

What is claimed is:

1. A stent, comprising:
(a) A substrate; and
(b) An agent on the substrate that prevents or treats, by disruption, elimination, or reduction, pathologic vasa vasorum and/or has anti-angiogenic properties.

2. The stent of claim 1, wherein the agent is selected from the group consisting of bevacizumab, Vitaxin®, angiostatin, and endostatins or a combination thereof.

3. The stent of claim 2, wherein the agent is bevacizumab.

4. The stent of claim 1, wherein the agent is on the substrate in an amount from about 0.01 micrograms/mm² to about 10 micrograms/mm².

5. The stent of claim 1 further comprising at least one anti-proliferative and/or anti-inflammatory agent on the substrate.

6. The stent of claim 5, wherein the at least one anti-proliferative and/or anti-inflammatory agent is selected from the group consisting of rapamycin, everlomius, biolimus, ABT 75, dexamethasone, paclitaxel, and their salts, prodrugs, derivatives and analogs, or a combination thereof.

7. The stent of claim 1, wherein the substrate is a balloon-expandable stent.

8. The stent of claim 1, wherein the substrate is a self-expandable stent.

9. The stent of claim 7, wherein the balloon-expandable stent is composed of a material selected from the group consisting of metal, an alloy, a polymer, a biodegradable alloy, biodegradable polymer, or a combination thereof.

10. The stent of claim 8, wherein the self-expandable stent is composed of a material selected from the group consisting of metal, an alloy, nitinol, a polymer, a biodegradable alloy, biodegradable polymer, or a combination thereof.

11. The stent of claim 1, wherein the stent has a diameter of about 2 mm to about 12 mm.

12. A method of treating vulnerable (unstable) and/or stable atherosclerotic plaque by disrupting pathologic vasa vasorum of the atherosclerotic plaque, comprising the steps of:
(a) Providing a stent comprising an agent that prevents or treats, by disruption, elimination, or reduction, pathologic vasa vasorum and/or has anti-angiogenic properties on the stent; and
(b) Positioning the stent at a plaque area.

13. The method of claim 12, wherein the agent is selected from the group consisting of bevacizumab, Vitaxin®, angiostatin, and endostatins or a combination thereof.

14. The method of claim 13, wherein the agent is bevacizumab.

15. The method of claim 12, wherein the agent is on the substrate in an amount from about 0.01 micrograms/mm² to about 10 micrograms/mm².

16. The method of claim 12, wherein the stent further comprise at least one anti-proliferative and/or anti-inflammatory agent on the stent.

17. The method of claim 16, wherein the at least one anti-proliferative and/or anti-inflammatory agent is selected from the group consisting of rapamycin, everlomius, biolimus, ABT 75, dexamethasone, paclitaxel, and their salts, prodrugs, derivatives and analogs or a combination thereof.

18. The method of claim 12, wherein the stent has a diameter of about 2 mm to about 12 mm.

19. The method of claim 12, wherein the stent is a balloon-expandable stent.

20. The method of claim 12, wherein the stent is a self-expandable stent.

21. The method of claim 19, wherein the balloon-expandable stent is composed of a material selected from the group consisting of metal, an alloy, a polymer, a biodegradable alloy, biodegradable polymer, or a combination thereof.

22. The method of claim 12, wherein the stent is delivered to the plaque area and positioned using a catheter

23. The method of claim 22, wherein the catheter further comprises at least one temperature sensor.

24. A method of treating vulnerable (unstable) and/or stable atherosclerotic plaque by disrupting pathologic vasa vasorum of the atherosclerotic plaque, comprising administering an agent that prevents or treats, by disruption, elimination, or reduction, pathologic vasa vasorum and/or has anti-angiogenic properties locally to the plaque area via a local delivery catheter system.

25. The method of claim 24, wherein delivery of the agent is intramural into the vessel wall, periadventitial, or intraluminal.

26. The method of claim 25, wherein the agent is delivered into periadventitial areas via nipple, needle or needles comprising catheters.

27. The method of claim 25, wherein intramural delivery is performed via a special local delivery catheter system.

28. The method of claim 26, wherein the special local delivery catheter system is selected from the group consisting of pressure mediated diffusion systems, convection driven delivery systems, iontophoretic-mediated diffusion systems, and active injector systems.

29. The method of claim 25, wherein the drug is delivered intraluminally into the lumen of a diseased artery by an infusion local delivery catheter system.

30. The method of claim 25, wherein an irrigator catheter is used to deliver the agent on a surface of a plaque area.

31. The method of claim 25, wherein a balloon catheter is coated with the agent to deliver the agent to a target area.

32. A method of treating vulnerable (unstable) and/or stable atherosclerotic plaque by disrupting pathologic vasa vasorum of the atherosclerotic plaque, comprising denervating an atherosclerotic vessel with one or more percutaneous catheter systems.

33. A method of treating vulnerable (unstable) and/or stable atherosclerotic plaque by disrupting pathologic vasa vasorum of the atherosclerotic plaque, comprising systemically administering an agent that prevents or treats, by disruption, elimination, or reduction, pathologic vasa vasorum and/or has anti-angiogenic properties.

34. The method of claim 33, wherein the systemic administration of the agent is nanoparticle-based.

35. A method of treating vulnerable (unstable) and/or stable atherosclerotic plaque by disrupting pathologic vasa vasorum of the atherosclerotic plaque, comprising two or more of the following steps:
(a) positioning a stent at a plaque area;
(b) administering an agent that prevents or treats, by disruption, elimination, or reduction, pathologic vasa vasorum and/or has anti-angiogenic properties locally to the plaque area via a local delivery catheter system;
(c) denervating an atherosclerotic vessel with one or more percutaneous catheter systems; and/or
(d) systemically administering an agent that prevents or treats, by disruption, elimination, or reduction, pathologic vasa vasorum and/or has anti-angiogenic properties.

36. The method of claim 35, wherein the stent comprises an agent that prevents or treats, by disruption, elimination, or reduction, pathologic vasa vasorum and/or has anti-angiogenic properties.
